(19) United States
(12) Patent Application Publication
(10) Pub. No.: US 2023/0321140 A1
(43) Pub. Date: Oct. 12, 2023

(54) MULTIPLE MEDIATORS TARGETED MULTIFUNCTIONAL NANOPARTICLES AND USES THEREOF

(71) Applicant: The Trustees of Columbia University in the City of New York, New York, NY (US)

(72) Inventors: Kam W. LEONG, New York, NY (US); Liu FENG, New York, NY (US); Dan SHAO, New York, NY (US)

(21) Appl. No.: 18/298,042

(22) Filed: Apr. 10, 2023

Related U.S. Application Data
(60) Provisional application No. 63/329,124, filed on Apr. 8, 2022.

(51) Int. Cl. A61K 33/30; A61K 31/7034; A61K 31/7036; A61P 31/04; A61P 39/06; A61P 37/02; G01N 33/68

(52) U.S. Cl. CPC A61K 33/30; A61K 31/7034; A61K 31/7036; A61P 31/04; A61P 39/06; A61P 37/02; G01N 33/6863; G01N 2333/525

(57) ABSTRACT

The present subject matter provides a series of multifunctional anti-inflammatory agents comprising tannic acid, Zn²⁺, and different amounts of gentamicin (TA-Zn-Gen NPs) to effectively improve sepsis treatment through five modes of anti-sepsis activity: (1) bound cfDNA with high affinity and inhibited cfDNA-induced activation of TLRs and nuclear factor kappa B (NF-κB) signaling; (2) inhibited activated macrophage-induced macrophage recruitment; (3) scavenged ROS and reduced ROS-induced DNA damage and cell death; (4) inhibited NO production induced by bacterial LPS; and (5) provided potent antibacterial activity.

### CROSS-REFERENCE TO RELATED APPLICATION

This application claims benefit of U.S. Provisional Application No. 63/329,124 filed Apr. 8, 2022, the contents of which are hereby incorporated by reference.

### GOVERNMENT LICENSE RIGHTS

This invention was made with government support under grant no. AR073935, awarded by the National Institutes of Health (NIH). The government has certain rights in the invention.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD

The present subject matter discloses multifunctional nanoparticles (NPs) comprising tannic acid, zinc ions (Zn²⁺), gentamicin (TA-Zn-Gen NPs) that are useful as anti-inflammatory agents to target multiple mediators of sepsis and improve sepsis treatment.

### BACKGROUND

Sepsis is a life-threatening systemic inflammatory response to fungal, bacterial, or viral infection.^{1,2} Despite advances in sepsis treatment, the mortality rate of sepsis remains high and over eight million people die annually due to sepsis.³ Recent efforts to develop more effective treatments of sepsis have focused on its pathogenesis,^{4,5} and multiple mediators of sepsis that are essential to its onset and progression have been identified.⁶⁻⁸ During the progression of sepsis, damage-associated molecular patterns (DAMPs) released by host cells and pathogen-associated molecular patterns (PAMPs) initiate inflammatory reactions that can cause a “cytokine storm”, multiple organ failure, and death.^{9,10} Inflammatory circulating cell-free DNA (cfDNA)—nuclear or mitochondrial DNA released by damaged host cells—is one such DAMP, and activates immune cells via toll-like receptor (TLR) activation, eliciting a sterile inflammatory response.¹¹⁻¹³ Cationic nanoparticles have been explored for scavenging inflammatory anionic cfDNA to treat cfDNA-associated diseases,¹⁴⁻¹⁷ but their potential systemic cytotoxicity would limit their clinical use.¹⁸ Other mediators of sepsis include lipopolysaccharides (LPS) released from the membranes of infectious bacteria. LPS are potent PAMPs that activate immune cell TLRs, stimulating the production of nitric oxide (NO) and inflammatory cytokines.¹⁹⁻²¹ Antagonists of TLR activation can reduce inflammation in sepsis, but excessive TLR inhibition and systemic loss of TLR function can cause immune suppression and increase the risk of infection.²²⁻²⁴ Excessive production of reactive oxygen species (ROS) such as hydroxyl radicals (·OH) and superoxide radicals (O₂^{·-}) also plays an important role in sepsis.²⁵⁻²⁵ Increased ROS levels cause DNA damage resulting in loss of cell function, cell death, and release of cfDNA,²⁷ perpetuating systemic inflammation and leading to organ failure and death. Reducing ROS levels is thus critical in sepsis treatment.

Therapies that target only a single mediator of sepsis have failed to reduce its mortality rate.²⁸⁻³¹ Since multiple factors contribute dynamically to cause systemic inflammation during sepsis,³² we hypothesized that a multifunctional nanoparticle that targeted multiple factors simultaneously could achieve a greater anti-sepsis therapeutic effect.

### SUMMARY

The present subject matter now provides a series of multifunctional anti-inflammatory nanoparticle agents comprising tannic acid, Zn²⁺, and different amounts of gentamicin (TA-Zn-Gen NPs) to effectively improve sepsis treatment through five modes of anti-sepsis activity: (1) bound cfDNA with high affinity and inhibited cfDNA-induced activation of TLRs and nuclear factor kappa B (NF-κB) signaling; (2) inhibited activated macrophage-induced macrophage recruitment; (3) scavenged ROS and reduced ROS-induced DNA damage and cell death; (4) inhibited NO production induced by bacterial LPS; and (5) provided potent antibacterial activity. The nanomaterials are less toxic than previously reported cfDNA-scavenging materials.

One embodiment provides a method of inhibiting activation of a pattern recognition receptor (PRR) to treat an inflammatory or immune response which is induced by the PRR, where the method comprises administering to a patient in need thereof a nanoparticle agent comprising tannic acid, Zn¹⁺ and gentamicin in an amount and under conditions such that the inhibition of the activation is effected, wherein the PRR is activated by a nucleic acid and the agent binds the nucleic acid. Preferably, the PRR is a cytoplasmic PRR or a TLR. In certain embodiments, the PRR is TLR3, TLR9, or both. In the present method, the multifunctional nanoparticles bind the nucleic acid in a manner that is independent of the sequences, structure or chemistry of the nucleic acid and the agent is cationic nanoparticles.

Another embodiment provides a method of inhibiting activated immune cell induced immune cell migration in the inflammation site. Preferably, the immune cell is macrophage or neutrophil. In one embodiment, the macrophage is RAW 264.7. In the present method, the multifunctional nanoparticles inhibit activated macrophage induced macrophage recruitment in transwell study and peritoneal cavity of septic mice.

Another embodiment provides a method of inhibiting cell death and inhibiting cfDNA release from cell which is induced by the overexpressed ROS in the inflammation site. Preferably, the ROS include all the ROS generated in the cell. In one embodiment, the ROS is hydroxyl radicals (·OH) and superoxide radicals (O₂^{·-}). In the present method, the multifunctional nanoparticles scavenge ·OH and O₂^{·-} in a dose-dependent manner.

Another embodiment provides a method of inhibiting NO generation from cell which is induced by the LPS in the inflammation site. Preferably, LPS is derived from gram-negative bacteria. In one embodiment, the LPS is derived from Escherichia coli. In the present method, the multifunctional nanoparticles inhibit LPS-induced NO generation in a dose-dependent manner.

Another embodiment provides a method of inhibiting inflammatory response which is induced by the bacteria, wherein the effective component is gentamicin and tannic acid. Preferably, the bacteria are gram-negative bacteria. In one embodiment, the bacteria are Escherichia coli. In the present method, the multifunctional nanoparticles display greater anti-bacterial effect than the gentamicin and tannic acid at the same dose.

Advantageously, the present method further comprises the step of exposing the patient to a nucleic acid/ROS/LPS/bacteria prior to administering the agent. Generally, the patient was already exposed to a nucleic acid/ROS/LPS/bacteria prior to administering of the agent.

The administration of the agent results in a reduction in the acute inflammatory response in the patient. The administration of the agent can be used to treat patients suffering from a disease selected from the group consisting of rheumatoid arthritis, spinal cord injury, psoriasis, systemic lupus erythematosus, inflammatory bowel disease, traumatic brain injury, an infectious disease, a cardiovascular disease, cancer, bacterial sepsis, multiple sclerosis, chronic obstructive pulmonary disease, and obesity.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Further features of the inventive concept, its nature and various advantages will be more apparent from the following detailed description, taken in conjunction with the accompanying figures:

FIGS. 1A-F show (A) average particle Size, (B) polydispersity index (PDI), and (C) zeta potential of TA-Zn-Gen NPs with increasing gentamicin content. TA:Gen weight ratio=(1) 1:0.125, (2) 1:0.25, (3) 1:0.5, (4) 1:1, (5) 1:2. A sharp increase in particle size and PDI and a change from net negative to positive surface charge occurred between a TA:Gen weight ratio of up to 1:0.5 (TA-Zn-Gen 1 to Gen 3) and 1:1 or higher (TA-Zn-Gen 4 and Gen 5). (D) ICP-MS and element analysis, (E) FTIR spectra, and (F) X-ray diffraction spectra of TA-Zn-Gen NPs 1-3.

FIGS. 2A-B show DNA binding affinity of NPs at different NP:DNA mass ratios (see legend) in TE buffer without Fetal Bovine Serum (FBS) (A) and with 10% FBS (B). [Calf thymus DNA]=0.5 μg/mL. (C) Cytotoxicity of NPs to RAW 264.7 cells. (D) Colocalization of FITC-labeled TA-Zn-Gen NPs and Cy5-labeled CpG in lysosomes (CLSM images). Scale bar, 10 μm.

FIGS. 3A-I show activation of HEK-Blue (A) hTLR9, (B) hTLR3 and (C) hTLR4 cells in the absence of FBS after different treatments for 24 h. Activation of HEK-Blue (D) hTLR9, (E) hTLR3 and (F) hTLR4 cells in the presence of FBS after different treatments for 24 h. The legend indicates NPs:agonist mass ratios. (G) Schematic of inhibition of nucleic acid-induced TLR activation by TA-Zn-Gen NPs. (H) TNF-α mRNA and (I) TNF-α cytokine generated by macrophages after different treatments for 24 h.

FIG. 4 illustrates inhibition of activated macrophage-induced macrophage migration measured using a transwell assay.

FIGS. 5A-G show (A) ·OH and (B) O₂^{·-} scavenging capability of the TA-Zn-Gen NPs. In panel A:T, TMB; H, H₂O₂; C, Cu²⁺. (C) Intracellular ROS imaging of macrophages after treatments. BF, bright field. Scale bar, 100 μm. (D) Intracellular ROS level evaluated by measuring 2′-7′dichlorofluorescein (DCF) fluorescence intensity with a multiwell plate reader. (E) NPs protection from ROS induced-DNA damage assessed using a plasmid nicking assay and agarose gel electrophoresis. (F) Phosphorylated histone variant H₂AX imaging of macrophages following different treatments. Scale bar, 100 μm. (G) Protective effect of NPs on ROS induced-cell death (CCK-8 assay). Data are expressed as mean±SD. Statistical comparisons of groups were performed using Student's t-test (*p<0.05, **p<0.01, ***p<0.001).

FIGS. 6A-F shows (A) Expression of iNOS mRNA in macrophages following different treatments. (B) Level of NO generated by macrophages following different treatments. (C-D) Antibacterial effect of TA-Zn-Gen NPs 1-3 after treatment for 6 h (C) and 12 h (D). (E-F) Antibacterial effect of free tannic acid, free gentamicin, and TA-Zn-Gen 3 NPs after treatment for 6 h (E) and 12 h (F). Data are expressed as mean±SD. Statistical comparisons of groups were performed using Student's t-test (*p<0.05, **p<0.01, ***p<0.001).

FIGS. 7A-D show (A) Schematic of CLP-induced sepsis model and treatment schedule. (B) Clinical score, (C) survival rate, and (D) body weight of mice in different treatment groups recorded for 5 consecutive days.

### DETAILED DESCRIPTION

Throughout this description, the preferred embodiments and examples provided herein should be considered as exemplar, rather than as limitations of the present technology.

We now provide a series of multifunctional TA-Zn-Gen NPs to effectively improve sepsis treatment through as many as five modes of anti-sepsis activity: (1) bound cfDNA with high affinity and inhibited cfDNA-induced activation of TLRs and nuclear factor kappa B (NF-KB) signaling; (2) inhibited activated macrophage-induced macrophage recruitment; (3) scavenged ROS and reduced ROS-induced DNA damage and cell death; (4) inhibited NO production induced by bacterial LPS; and (5) provided potent antibacterial activity. In certain embodiments, treatment of the patient provides at least one, or at least two, and as many as all five, of these activities. The nanomaterials are less toxic than other reported cfDNA-scavenging materials.

Multiple mediators of sepsis including cfDNA, ROS, LPS, immune cells and bacteria that are essential to its onset and progression have been identified. The sepsis mediated by one or multiple mediators can now be treated with TA-Zn-Gen NPs that possess up to five modes of anti-sepsis activity. The present technology provides a more effective and safe method to treat the sepsis by targeting multiple mediators.

During the progression of sepsis, damage-associated molecular patterns (DAMPs) released by host cells and pathogen-associated molecular patterns (PAMPs) initiate inflammatory reactions that can cause a “cytokine storm”, multiple organ failure, and death. Inflammatory circulating cell-free DNA (cfDNA)—nuclear or mitochondrial DNA released by damaged host cells—is one such DAMP, and activates immune cells via TLR activation, eliciting a sterile inflammatory response.

Cationic nanoparticles have been explored for scavenging inflammatory anionic cfDNA to treat cfDNA-associated diseases, but their potential systemic cytotoxicity would limit their clinical use. Other mediators of sepsis include LPS released from the membranes of infectious bacteria. LPS are potent PAMPs that activate immune cell TLRs, stimulating the production of NO and inflammatory cytokines. Antagonists of TLR activation can reduce inflammation in sepsis, but excessive TLR inhibition and systemic loss of TLR function can cause immune suppression and increase the risk of infection.

Excessive production of ROS such as ·OH and O₂^{·-} also plays an important role in sepsis. Increased ROS levels cause DNA damage resulting in loss of cell function, cell death, and release of cfDNA, perpetuating systemic inflammation and leading to organ failure and death. Reducing ROS levels is thus critical in sepsis treatment. Therapies that target only a single mediator of sepsis have failed to reduce its mortality rate. Since multiple factors contribute dynamically to cause systemic inflammation during sepsis, we hypothesized that a multifunctional nanoparticle that targeted multiple factors simultaneously could achieve a greater anti-sepsis therapeutic effect.

The three kinds of multifunctional TA-Zn-Gen NPs (TA-Zn-Gen 1, TA-Zn-Gen 2 and TA-Zn-Gen 3 NPs) that comprise or consist of tannic acid, Zn²⁺, and gentamicin were synthesized by varying the introduced amount of gentamicin using a simple, low-cost, scalable one-pot process at ambient temperature.

The present subject matter also provides a method of controlling (inhibiting or preventing) autoimmune and/or inflammatory responses associated with activation of multiple PRRs, including TLR3 and TLR9. Multifunctional TA-Zn-Gen NPs possessed a net negative surface charge (favorable for avoiding NPs cytotoxicity) but still bound cfDNA with high affinity. TA-Zn-Gen 1, TA-Zn-Gen 2 and TA-Zn-Gen 3 NPs can efficiently inhibit the activation of an inflammatory response against the nucleic acids. In one embodiment, TA-Zn-Gen 1, TA-Zn-Gen 2 and TA-Zn-Gen 3 NPs can effectively inhibit the activation of multiple nucleic acid sensing PRRs, including TLR3, TLR9 in human embryonic kidney 293 cells. In another embodiment, TA-Zn-Gen 1, TA-Zn-Gen 2 and TA-Zn-Gen 3 NPs can effectively inhibit the TNF-a release from RAW 264.7 cells induced by pathogenic nucleic acids.

One embodiment provides a method of inhibiting activated immune cell induced immune cell migration in the inflammation site. Preferably, the immune cell is macrophage or neutrophil. In one embodiment, the macrophage is RAW 264.7. In the present method, the TA-Zn-Gen 1, TA-Zn-Gen 2 and TA-Zn-Gen 3 NPs inhibit activated macrophage induced macrophage recruitment in transwell study and peritoneal cavity of septic mice.

The technology provides a method of inhibiting cell death and inhibiting cfDNA release from cell which is induced by the overexpressed ROS in the inflammation site. Preferably, the ROS include all the ROS generated in the cell. In one embodiment, the ROS is hydroxyl radicals (·OH) and superoxide radicals (O₂^{·-}). In the present method, the TA-Zn-Gen 1, TA-Zn-Gen 2 and TA-Zn-Gen 3 NPs scavenge ·OH and O₂^{·-} in a dose-dependent manner.

Another embodiment provides a method of inhibiting NO generation from cell which is induced by the LPS in the inflammation site. Preferably, the LPS is derived from gram-negative bacteria. In one embodiment, the LPS is derived from Escherichia coli. In the present method, the TA-Zn-Gen 1, TA-Zn-Gen 2 and TA-Zn-Gen 3 NPs inhibit LPS-induced NO generation in a dose-dependent manner.

Another embodiment provides a method of inhibiting inflammatory response which is induced by the bacteria, wherein the effective component is gentamicin and tannic acid. Preferably, the bacteria are gram-negative bacteria. In one embodiment, the bacteria are Escherichia coli. In the present method, the TA-Zn-Gen 1, TA-Zn-Gen 2 and TA-Zn-Gen 3 NPs display greater anti-bacterial effect than the gentamicin and tannic acid at the same dose.

The TA-Zn-Gen 1, TA-Zn-Gen 2 and TA-Zn-Gen 3 NPs could also be applied for treating broad diseases/disorders including rheumatoid arthritis, spinal cord injury, psoriasis, systemic lupus erythematosus, inflammatory bowel disease, traumatic brain injury, an infectious disease, a cardiovascular disease, cancer bacterial sepsis, multiple sclerosis, chronic obstructive pulmonary disease, and obesity.

The TA-Zn-Gen 1, TA-Zn-Gen 2 and TA-Zn-Gen 3 NPs can be administered to the patient via any route such that effective levels are achieved in, for example, the bloodstream. The optimum dosing regimen will depend, for example, on the cationic nucleic acid scavenger, the patient and the effect sought. Typically, the TA-Zn-Gen 1, TA-Zn-Gen 2 and TA-Zn-Gen 3 NPs will be administered orally, IV, IM, IP or SC. the TA-Zn-Gen 1, TA-Zn-Gen 2 and TA-Zn-Gen 3 NPs can also be administered, for example, directly to a target site.

The TA-Zn-Gen 1, TA-Zn-Gen 2 and TA-Zn-Gen 3 NPs, or pharmaceutically acceptable salts thereof, can be formulated with a carrier, diluent or excipient to yield a pharmaceutical composition. The precise nature of the compositions will depend, at least in part, on the nature of the nucleic acid binding agent and the route of administration. Optimum dosing regimens can be readily established by one skilled in the art and can vary with the nucleic acid binding agent, the patient and the effect sought. For example, depending on the disease and the severity of the disease, one skilled in the art will readily determine an appropriate or optimal dosing regimen for the patient. For example, the dose may depend in part on the expected effect of the composition, while the frequency of dosing may depend in part on the in vivo clearance of the composition, which itself will depend in part on the route of administration.

It will be appreciated that the present treatment methods are useful in the fields of both human medicine and veterinary medicine. Thus, the patient (subject) to be treated can be a mammal, preferably a human. For veterinary purposes the subject can be, for example, a farm animal such as a cow, pig, horse, goat or sheep, or a companion animal such as a dog or a cat.

### EXAMPLE

The following examples illustrate the benefits and advantages of the present technology.

### Example 1

### Generation of TA-Zn-Gen NPs

Tannic acid solution, Zn (NO₃)₂·6H₂O solution, and different amounts of gentamicin solution were continuously added to Milli-Q water. The mixture was stirred at room temperature for 10 min, then the pH was adjusted to 5.2 using 10 M NaOH solution, and stirring was continued for another 20 min. The complex was then centrifuged at 8000 rpm for 5 min, and the collected precipitate was washed with Milli-Q water for three times. The precipitate was lyophilized with trehalose as a cryoprotectant for further use.

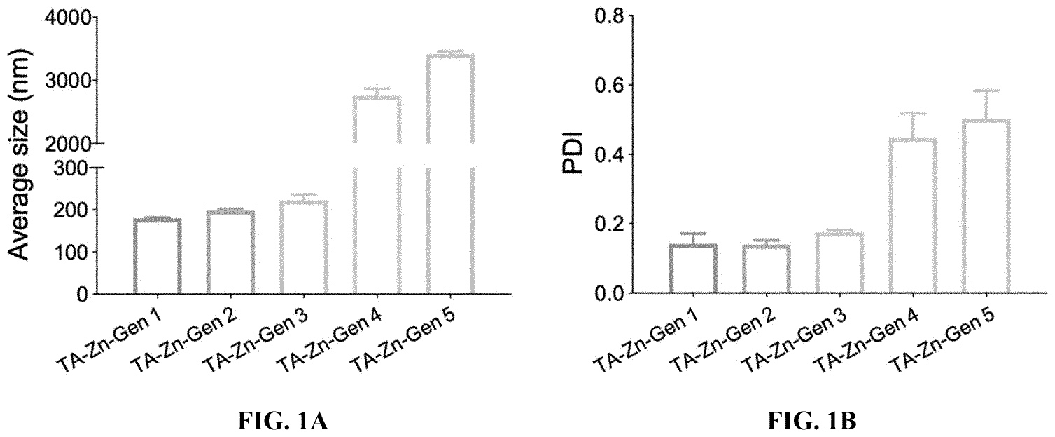

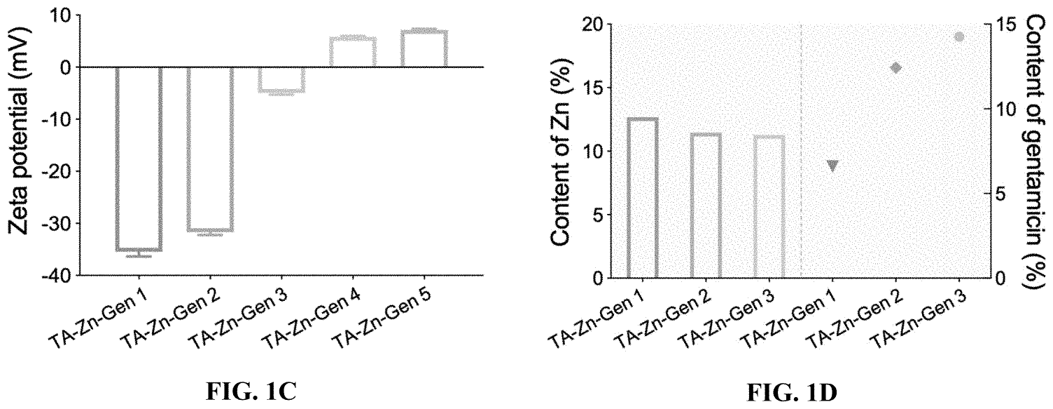

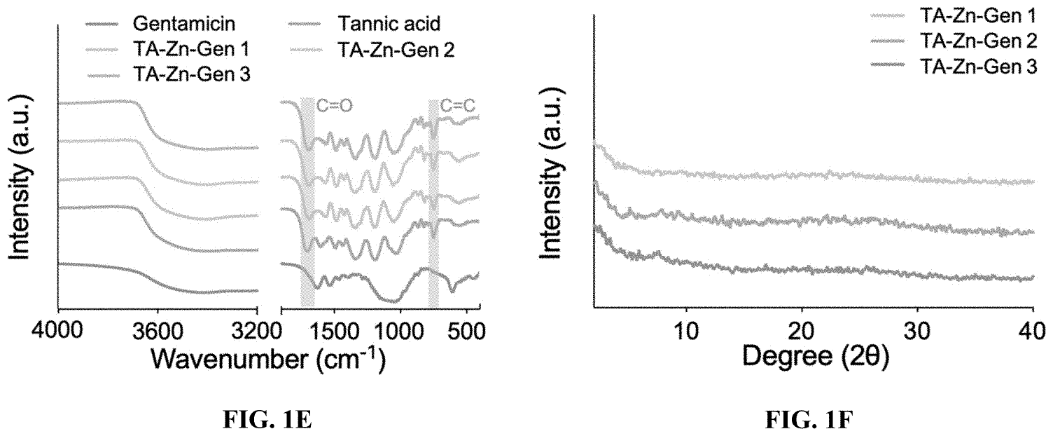

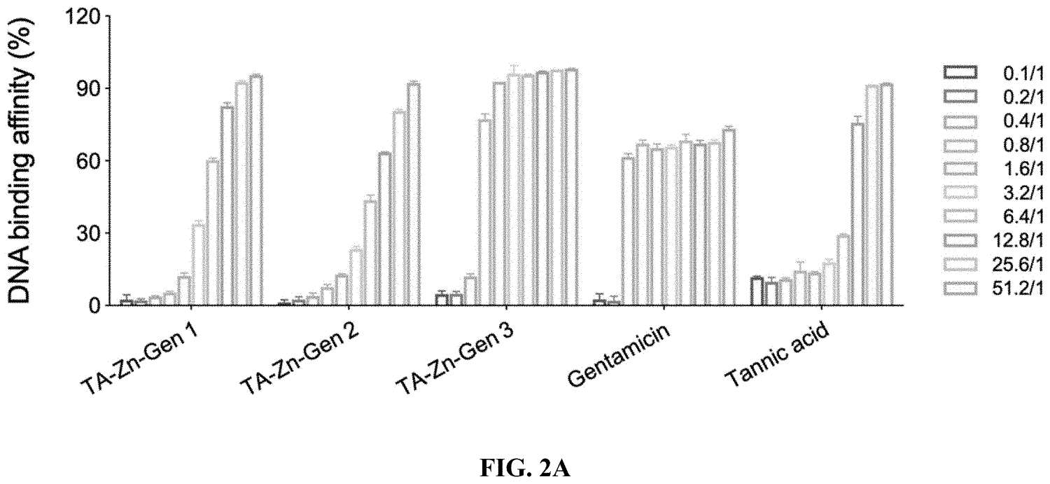

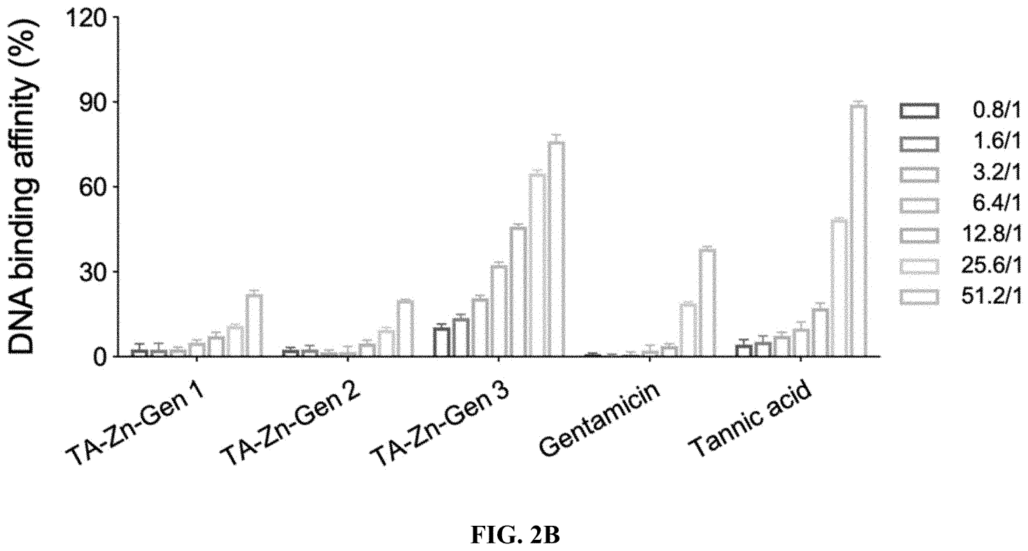

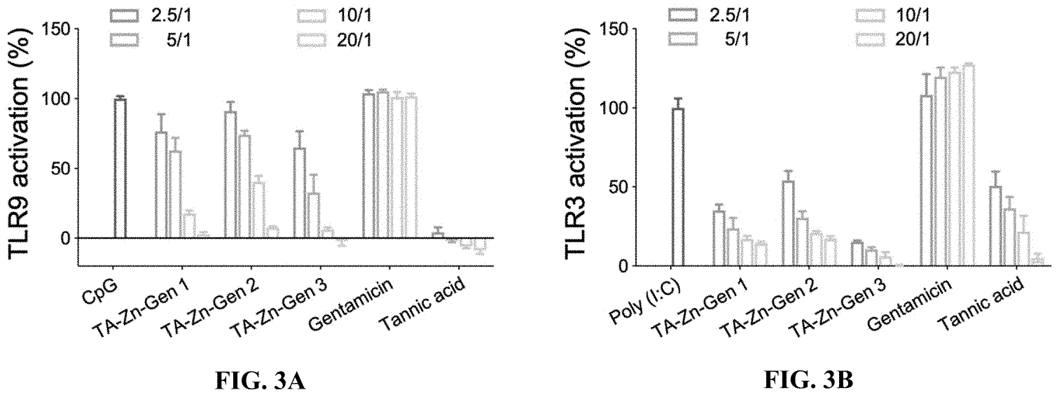

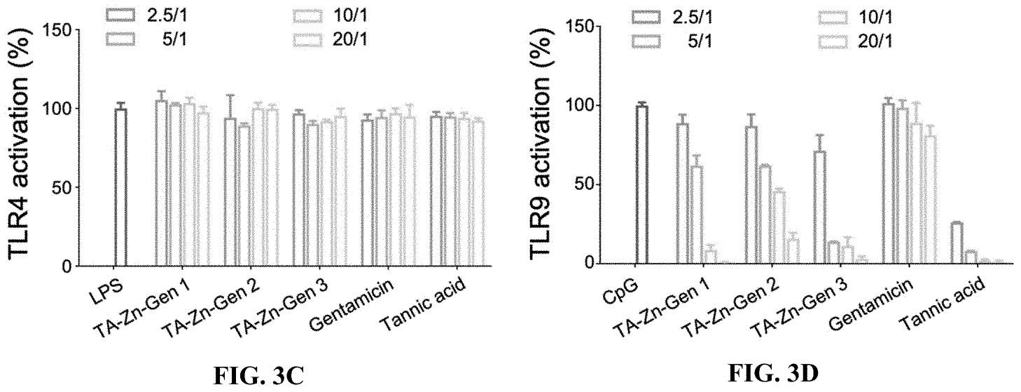

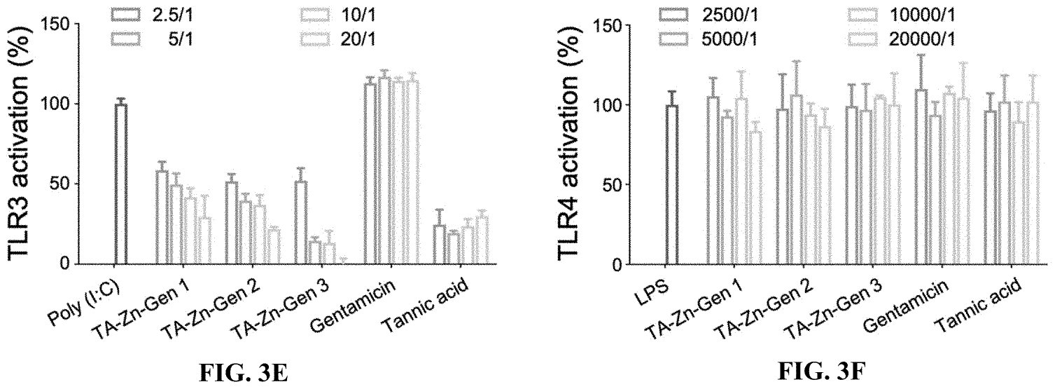

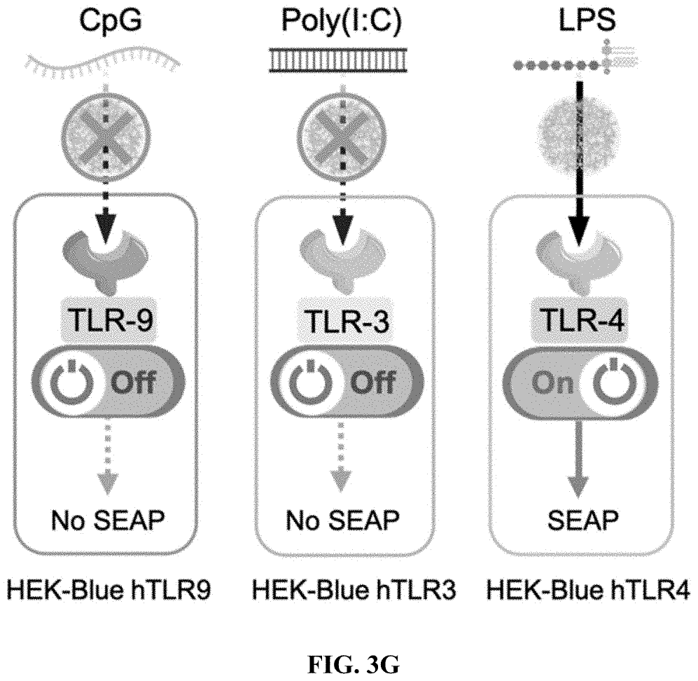

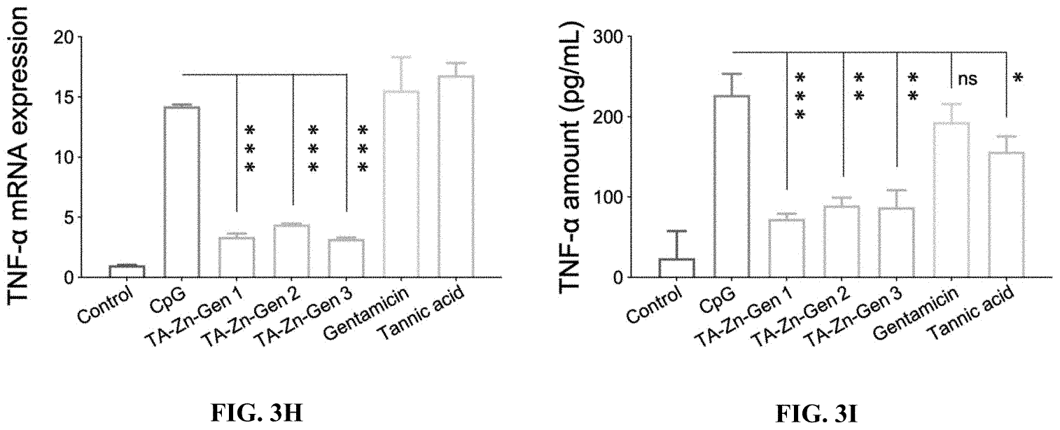

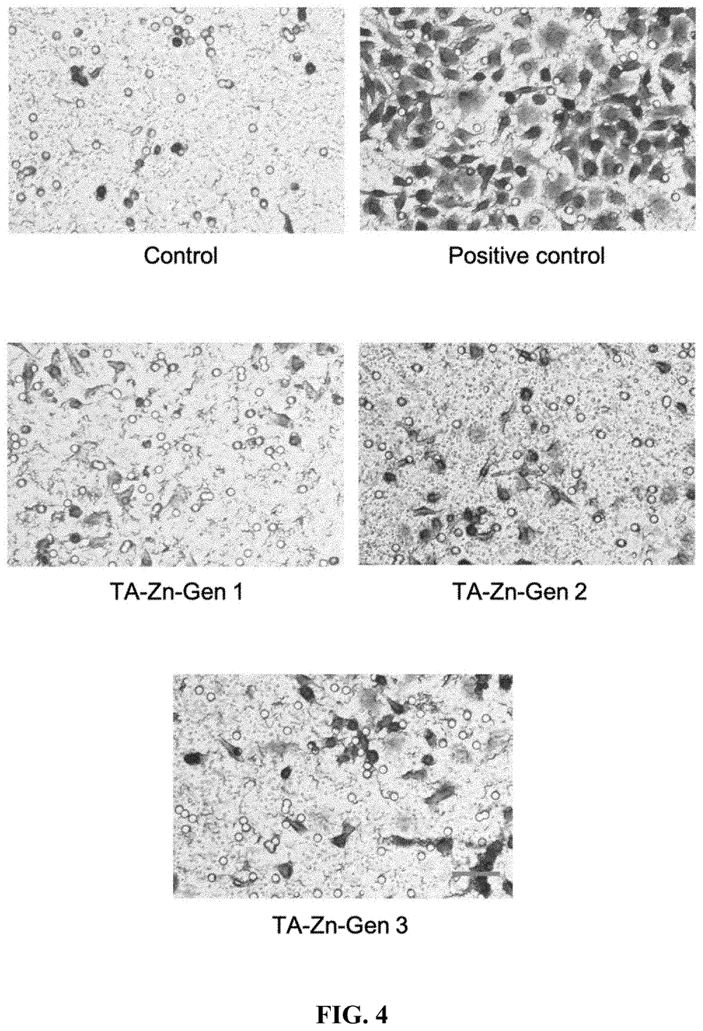

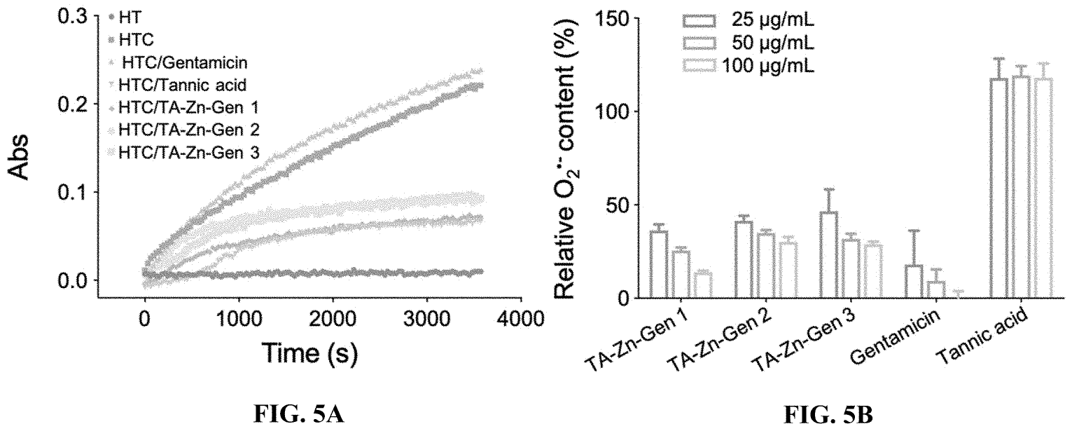

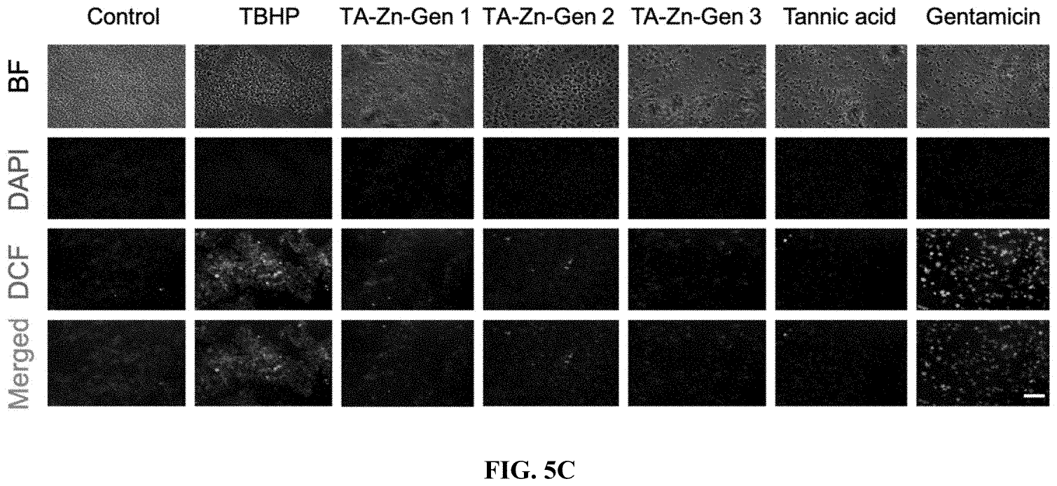

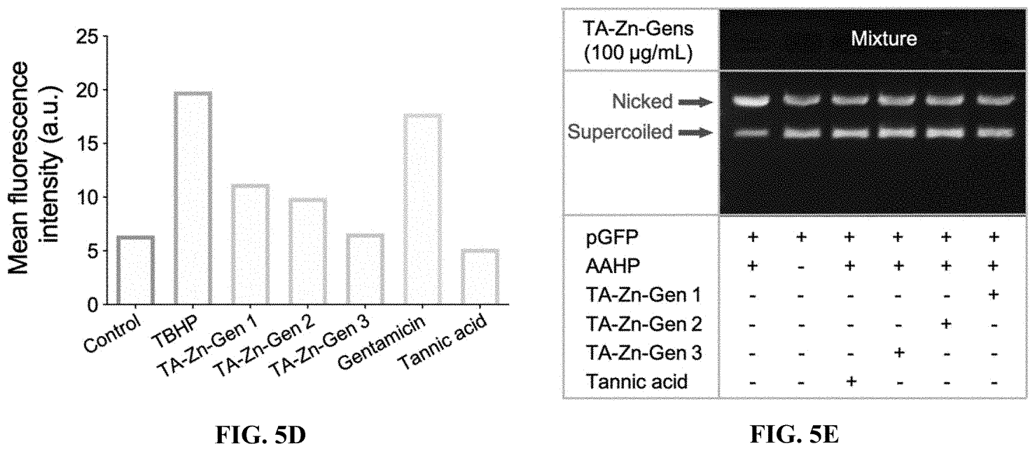

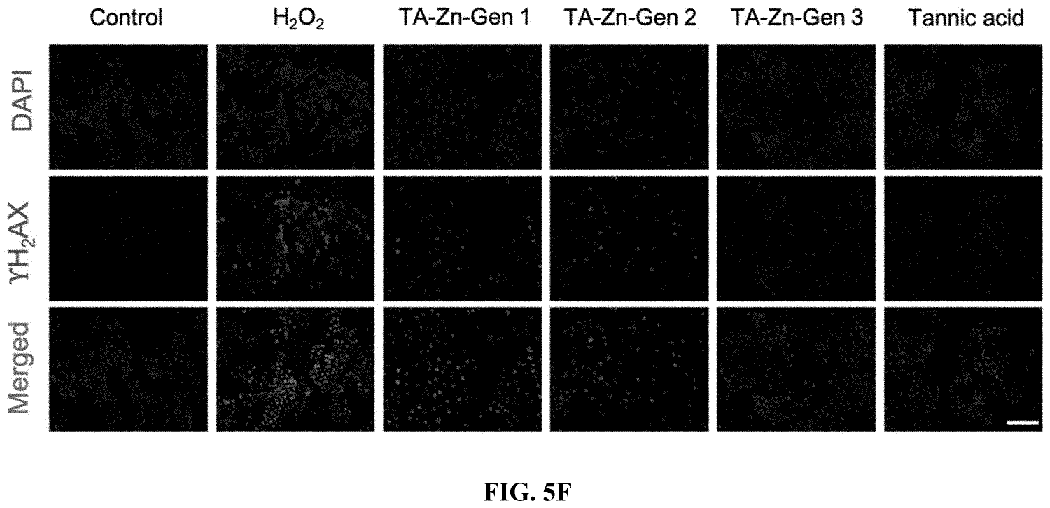

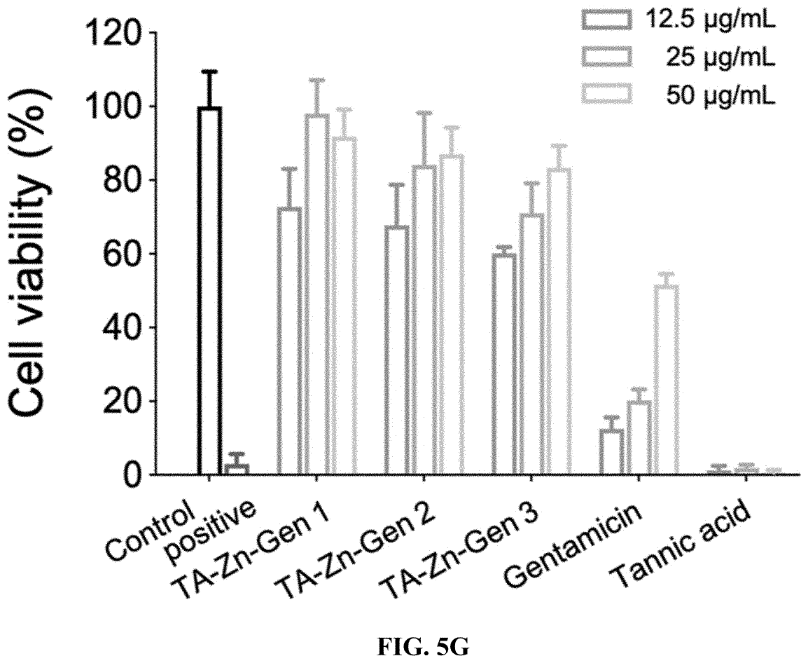

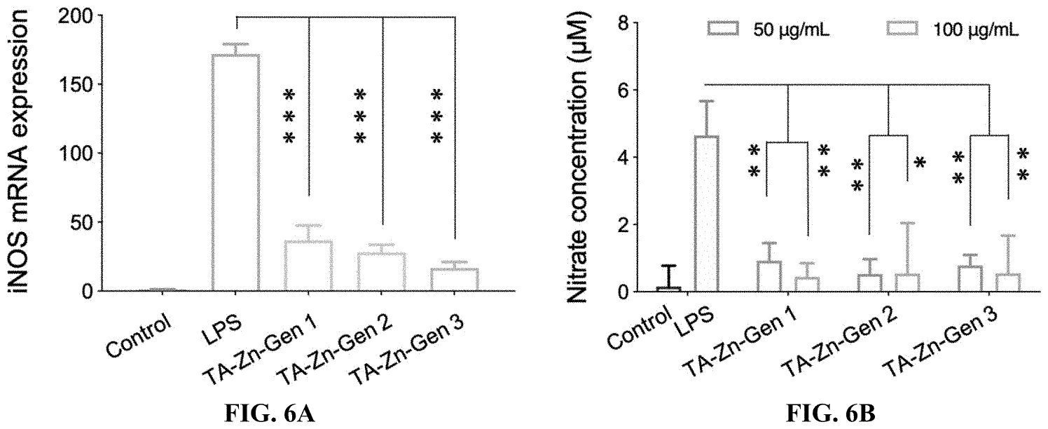

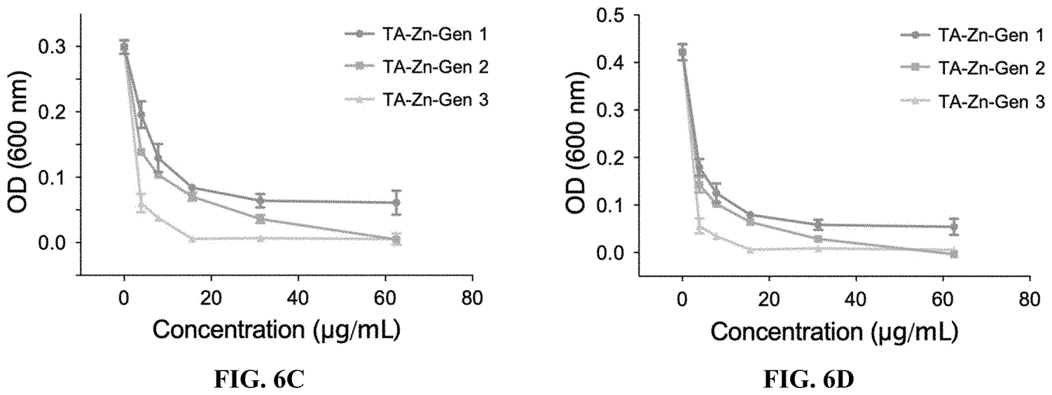

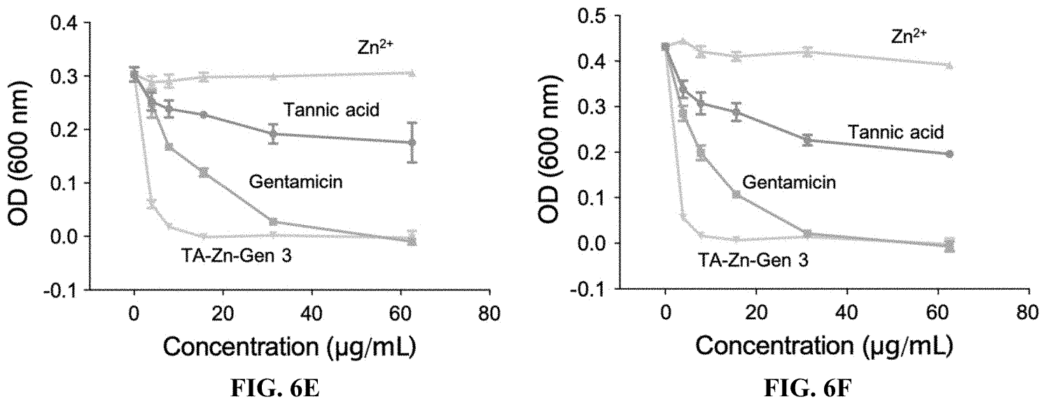

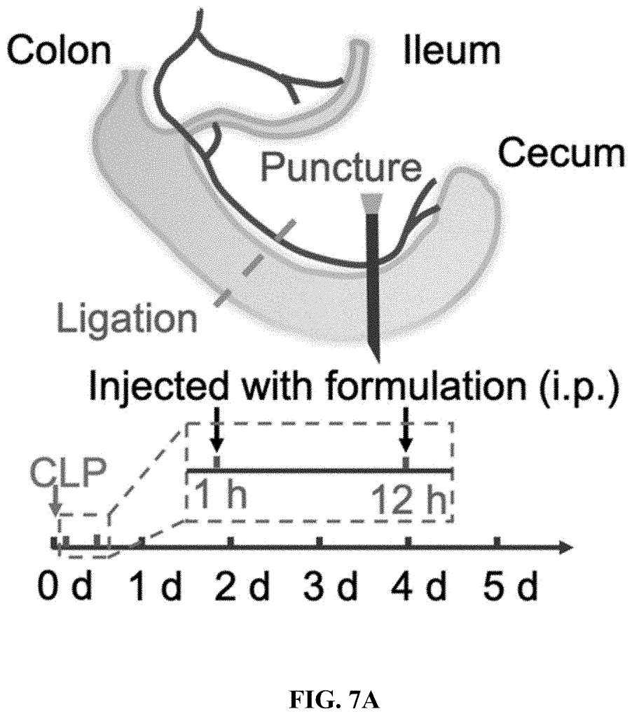

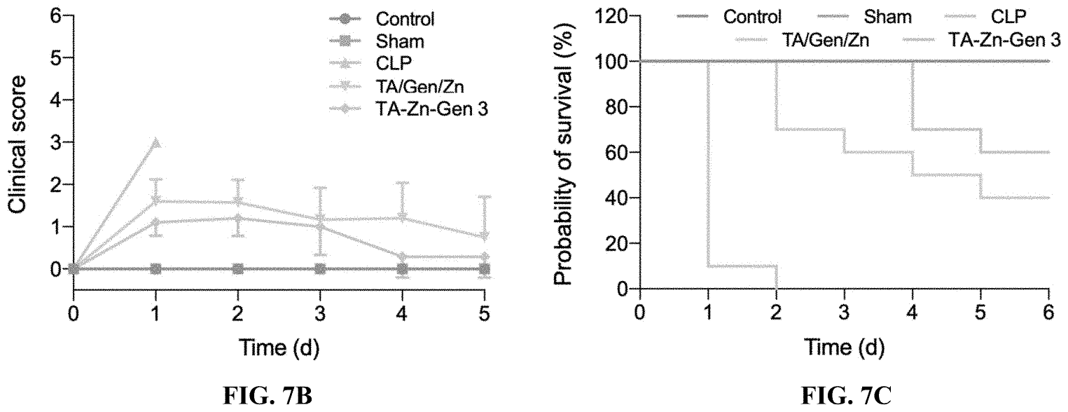

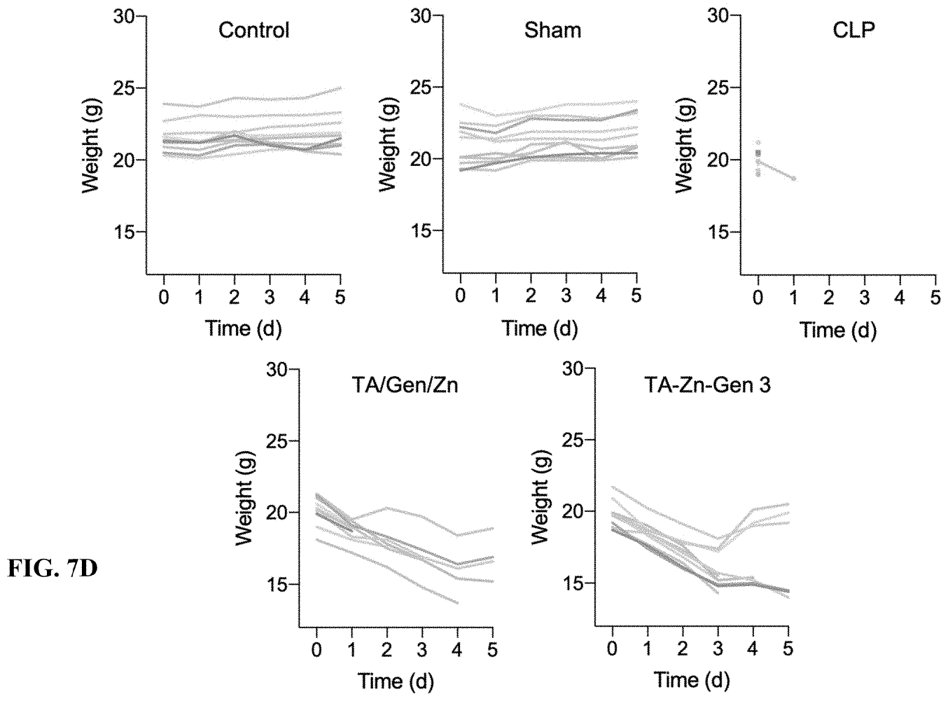

TA-Zn-Gen NPs were characterized in terms of their composition and physiochemical properties (FIG. 1), DNA binding affinity (FIG. 2), inhibition of cfDNA-induced TLR activation and inflammatory cytokine release (FIG. 3), inhibition on activated microphage induced microphage migration (FIG. 4), ROS scavenging capability (FIG. 5), inhibition on LPS induced NO production and anti-bacterial effect (FIG. 6), and anti-sepsis therapeutic activity in a cecal ligation and puncture (CLP)-induced sepsis model in vivo (FIG. 7).

As shown in FIGS. 1A, 1B and 1C, the size, polydispersity index, and zeta potential of the NPs increased with increasing gentamicin content. Intriguingly, when the TA:Gen weight ratio was increased from 1:0.5 to 1:1, the NPs size increased sharply from ˜200 nm to over 2 μm in diameter, and the surface charge flipped from negative to positive. Since anionic NPs are favorable for prolonging blood circulation time and enhancing accumulation in inflamed sites, we selected the three smaller, anionic NPs for further investigation. Element analysis confirmed the presence of nitrogen (due to gentamicin) in the NPs (FIG. 1D), and inductively coupled plasma mass spectroscopy (ICP-MS) confirmed the presence of zinc (FIG. 1D). C═O and C═C characteristic peaks of tannic acid were observed in the Fourier-transform infrared spectroscopy (FTIR) spectra of the NPs (FIG. 1E), confirming the presence of tannic acid. No crystalline peak was observed in X-ray diffraction (XRD) spectra (FIG. 1F), indicating an amorphous structure.

### Example 2

### DNA Binding Affinity of TA-Zn-Gen NPs

Quant-iT PicoGreen (12.5 μL) and calf thymus DNA (5 mg/mL, 25 μL) were mixed with 1×TE buffer (10 mL) in the dark. NPs at different concentrations in 100 μL together with 100 μL of the above solution were added to a 96-well black plate and incubated at 37° C. for 30 min. The fluorescence intensity of the Picogreen-DNA complex at 520 nm was measured with a multiwell plate reader via excitation at 490 nm.

As shown in FIG. 2A, despite their net negative surface charge, TA-Zn-Gen NPs 1-3 exhibited high DNA binding affinity. TA-Zn-Gen NPs with increasing gentamicin content displayed increasing DNA binding affinity, possibly due to electrostatic interactions between DNA and gentamicin. Interestingly, free tannic acid also exhibited DNA binding, possibly due to hydrogen bonding between tannic acid and the phosphate backbone of DNA. The addition of 10% FBS reduced DNA binding in all groups, but the competitive interactions due to serum proteins were overcome by increasing the amount of NPs (FIG. 2B).

### Example 3

### Inhibition of the Activation of Nucleic Acid Sensors by TA-Zn-Gen NPs

HEK-Blue hTLR cells were cultured and maintained in DMEM with 10% FBS and 1% penicillin-streptomycin. To evaluate NPs inhibition of TLR activation, HEK-Blue hTLR cells were seeded in a 96-well plate for 30 min then treated with 2 μL of agonist (CPG Bw006 or Poly (I:C), 1 mg/mL). After 20 min of incubation, 2 μL of NPs at different concentrations were introduced in a final volume of 200 μL. After 24 h, the supernatants were collected and mixed with Quanti-Blue. TLR activation associated with SEAP activity was determined with a multiwell plate reader by measuring the OD at 620 nm. The cell densities (in a 96 well plate) and agonists used were 8×104 cells/well and CpG Bw006 for HEK-Blue hTLR9 cells, 5×104 cells/well and poly (I:C) for HEK-Blue hTLR3 cells.

HEK-Blue hTLR3, hTLR4, and hTLR9 cells were constructed by co-transfecting the hTLR gene and an optimized secreted embryonic alkaline phosphatase (SEAP) reporter gene into HEK 293 cells. An IFN-β minimal promoter fused to five NF-κB and AP-1 binding sites was designed to control the expression of SEAP reporter gene. TLR agonist treatments initiate the expression of NF-κB and AP-1, which induce production of SEAP, which is detected by using Quanti-Blue reagent and measuring the optical density (OD) at 620 nm.

We tested three TLR agonists: (1) CpG Bw006 ssDNA oligonucleotide, a TLR9 ligand; (2) poly (I:C), a synthetic dsRNA analog that activates TLR3 signaling; and (3) LPS, a TLR4 ligand (FIG. 3A-F). Consistent with the previous nucleic acid binding results, the NPs inhibited CpG-induced activation of HEK-Blue hTLR9 cells and inhibited poly (I:C)-induced activation of HEK-Blue hTLR3 cells in a NPs dose-dependent manner, regardless of the presence or absence of FBS (FIGS. 3A, 3B, 3D, and 3E). An embodiment of the concept is illustrated in FIG. 3G. Together, these results demonstrate that the TA-Zn-Gen NPs specifically inhibit nucleic acid (DNA or RNA but not LPS)-induced TLR activation and downstream NF-κB signaling.

### Example 4

### Anti-Inflammatory Assays in Vitro

RAW 264.7 macrophages were seeded in a 96-well plate at 2×104 cells/well and after 30 min of incubation were treated with 2 μL of CpG Bw006 (1 mg/mL). After another 20 min, 2 μL of NPs at different concentrations were added in a final volume of 200 μL. After incubation for another 24 h, the supernatants were collected and assessed with a TNF-α ELISA Kit.

As shown in FIGS. 3H and 3I, Transcription of tumor necrosis factor-α (TNF-α) mRNA and expression of TNF-α cytokine both increased significantly after 24 h of co-culture of the CpG and the macrophages. Addition of NPs inhibited macrophage activation and TNF-α release, demonstrating an anti-inflammation effect due to DNA binding by the NPs.

### Example 5

### Inhibition of Activated Macrophage-Induced Macrophage Recruitment

A transwell assay was conducted to investigate the anti-macrophage migration activity of the TA-Zn-Gen NPs. RAW 264.7 cells were seeded in a 24-well plate at 2.5×105 cells/well and were allowed to adhere overnight in growth medium at 37° C., 5% CO₂. Then the cells were treated with CpG Bw006 for 4 h. Following repeatedly washes with PBS and adding growth medium without FBS, 8-μm pore polycarbonate transwell chambers containing resuspended RAW 264.7 cells (2.5×105 cells, 0.2 mL) were inserted into the wells. Next, the cells were treated with NPs for 24 h. For the negative control group, no CpG Bw006 or NPs were added; for the positive control group, only CpG Bw006 was added; for the experimental groups, both CpG Bw006 and NPs were added. After 24 h of incubation, the cells on the upper side of chamber were gently removed with a cotton swab. Subsequently, the cells on the lower side of chamber were fixed with 4% paraformaldehyde and stained with 0.1% crystal violet. The migration of cells was determined by microscopy and five images of migrated cells per high-power field were acquired and counted.

As shown in FIG. 4, macrophage accumulation in inflamed sites aggravates inflammation, and reducing activated macrophage-induced macrophage recruitment is a promising strategy for alleviating inflammation in sepsis.³⁹ CpG Bw006-activated RAW 264.7 macrophages recruited a large number of macrophages from the upper side of a transwell chamber to the lower side due to chemotaxis induced by release of attractants by activated macrophages. When TA-Zn-Gen NPs were added, macrophage migration was sharply reduced. Thus, the TA-Zn-Gen NPs not only inhibit nucleic acid-initiated TLR activation, but also inhibit activated macrophage-induced macrophage migration once macrophages are activated.

### Example 6

### ROS Scavenging Capability and Inhibition on ROS-Induced DNA Damage

·OH was generated by a Fenton reaction of CuSO₄ and H₂O₂. The amount of ·OH scavenged was calculated by measuring the characteristic absorbance of oxidized TMB (1 mM) at 650 nm in the presence of CuSO₄ (2 mM), H₂O₂ (5 mM), and NPs. O₂^{·-} were generated by mixing xanthine oxidase (0.05 U/mL) and xanthine (0.6 mM) in PBS at 37° C. for 40 min. NPs at different concentrations were added to the above solution and incubated for another 40 min, followed by adding hydroethidine at 0.5 mg/mL. The amount of O₂^{·-} scavenged was determined by measuring the fluorescent intensity of ethidium (oxidation product of hydroethidine by O₂^{·-}) at 610 nm with a multiwell plate reader via excitation at 470 nm.

For imaging ROS levels, RAW 264.7 cells were seeded in 12-well plates (1×105/well) and were allowed to adhere overnight in growth medium (37° C., 5% CO2). The cells were then treated with 1 mM TBHP for 4 h. Following three washes with PBS, the cells were treated with NPs for another 24 h. After staining with H₂DCFDA and DAPI, the cells were washed repeatedly with PBS and were imaged using a fluorescence microscope.

To investigate the protection effect on ROS-induced DNA damage, supercoiled pGFP plasmid DNA (0.05 μg/mL, 2 μL), NPs (100 μg/mL, 2 μL), AAPH (10 mM, 4 μL), and PBS (2 μL) were mixed and incubated at 37° C. for 1 h. After mixing with 1×loading buffer (2 μL), the samples were analyzed by electrophoresis in 1% agarose gels stained with ethidium bromide for 1 h (buffer: 45 mM Tris-borate, 1 mM EDTA, pH 8). After electrophoresis, the gels were illuminated with a UV transilluminator and photographed, and band intensities were analyzed using Image J software.

For imaging intracellular DNA damage level, RAW 264.7 cells were seeded on coverslips in a 96-well plate at 2×104 cells/well and were allowed to adhere overnight in growth medium at 37° C., 5% CO₂. The cells were then treated with 50 μM H₂O₂ and NPs (50 μg/mL) for 24 h. For the control group, no H₂O₂ and NPs were added; for the positive control group, only H₂O₂ was added. Following staining with DAPI and HSC DNA Damage Kit, the cells were washed with PBS and imaged with a fluorescence microscope.

As shown in FIG. 5A, The TA-Zn-Gen NPs markedly reduced the generation of oxidized TMB by OH in a NPs dose-dependent manner, indicating that the NPs scavenge hydroxyl radicals. Next, the oxidation of xanthine by xanthine oxidase was used to produce O₂^{·-}, and the O₂^{·-} scavenging ability of the TA-Zn-Gen NPs was characterized by measuring the fluorescence of ethidium, the product of hydroethidine oxidation by O₂^{·-} at 610 nm. The fluorescence intensity decreased with increasing NPs concentration (FIG. 5B), indicating elimination of O₂^{·-} by the NPs. The intracellular ROS level was then evaluated by using cell-permeant 2′,7′-dichlorodihydrofluorescein diacetate (H₂DCFDA) as an indicator. Bright green fluorescence was observed in tert-butyl hydroperoxide (TBHP)-treated cells upon stimulation (FIG. 5C), demonstrating the successful induction of oxidation pressure. With the introduction of the TA-Zn-Gen NPs, the fluorescence intensity in cells decreased significantly, indicating ROS scavenging by the NPs in vitro. Semi-quantitative analysis of these results using Image J software are shown in FIG. 5D.

Next, the effect of TA-Zn-Gen NPs on ROS-induced cell DNA damage was evaluated through a plasmid nicking assay. In this experiment, 2,2′-azobis(2-amidinopropane) dihydrochloride (AAPH) was used as an ROS source to convert supercoiled DNA into the nicked form. supercoiled DNA and nicked DNA possess different electrophoretic mobilities and can be separated by agarose gel electrophoresis. The supercoiled plasmid DNA strand was damaged by AAPH treatment, and a high fraction of the nicked form was observed in a gel electrophoretogram (FIG. 5E). In contrast, the formation of the nicked form decreased dramatically when TA-Zn-Gen NPs were added, demonstrating protection from ROS-induced DNA damage by the NPs. Next, NPs protection against cellular DNA damage was determined by measuring the amount of phosphorylation of the histone variant H₂AX. TA-Zn-Gen NPs-treated cells exhibited lower amounts of phosphorylated H₂AX (γH₂AX) than untreated cells (FIG. 5F), demonstrating the protective effect of the NPs on DNA integrity. Next, the protective effect of the TA-Zn-Gen NPs from ROS -induced cell death was investigated. The NPs reduced ROS-induced cell death in a dose-dependent manner (FIG. 4G). Collectively, these results show that the TA-Zn-Gen NPs scavenge intracellular ROS and prevent ROS-induced DNA damage and cell death. This blocking of ROS-related inflammatory macrophage signaling is an important anti-sepsis activity.

### Example 7

### Inhibition of LPS-Induced NO Generation and Anti-Bacterial Effect

RAW 264.7 cells were seeded in a 96-well plate at 4×104 cells/well and were allowed to adhere overnight in growth medium at 37° C., 5% CO₂. The cells were treated with LPS (10 ng/mL) and NPs (50 μg/mL or 100 μg/mL) for 24 h. For the negative control group, no LPS and NPs were added; for the positive control group, only LPS was added. Then the supernatants were collected and mixed with a NO Assay Kit, and nitric oxide generation was determined with a multiwell plate reader.

E. coli was used to investigate the anti-bacterial effect of NPs. E. coli diluted in Luria-Bertani liquid medium were seeded in a 96-well plate at a density of 10⁶/mL and were treated with NPs at different concentrations. After 6 h and 12 h of incubation with NPs at 37° C., the proliferation of bacteria was determined with a multiwell plate reader by measuring the OD at 600 nm.

As shown in FIGS. 6A and 6B, TA-Zn-Gen NPs could reduce LPS-stimulated iNOS mRNA expression and NO production in RAW 264.7 cells. TA-Zn-Gen NPs 1-3 (which have increasing gentamicin content) displayed increasing antibacterial activity at the same NPs concentration, indicating a gentamicin concentration-dependent antibacterial effect (FIG. 6C, D). Since TA-Zn-Gen 3 NPs exhibited the strongest antibacterial activity, we selected these NPs for comparisons with the free components of the NPs. Free tannic acid exhibited slight antibacterial activity at a high concentration, and free gentamicin showed a potent concentration-dependent antibacterial effect (FIG. 6E, F). Interestingly, TA-Zn-Gen 3 NPs exhibited greater antibacterial activity than free gentamicin and free tannic acid at the same dose, possibly due to a synergistic effect of gentamicin and tannic acid.

### Example 8

Therapeutic effect of cationic NPs in CLP model of sepsis

C57 mice (male, 6- or 8-week-old) were purchased from Liaoning Changsheng Biotechnology. All animal experiments were conducted according to the guidelines for laboratory animals established by the Animal Care and Use Committee of Northeast Normal University. The CLP-induced sepsis model was established as described previously.

Briefly, mice were anesthetized using an isoflurane anesthesia system, followed with abdominal hair shaving and disinfection. Next, the cecum was gently exteriorized after a 1 cm incision at the midline. After ligation with 4-0 silk at the designated position for severe grade sepsis, the cecum was punctured with a 21-gauge needle and the cecal content was extruded through the perforation. The cecum was then put back into the peritoneal cavity followed by stitching of the incision.

Mice in the CLP group underwent the CLP procedure described above without further treatment. Mice in the Sham group underwent only the abdominal laparotomy procedure without subsequent cecal ligation and puncture. In the TA/Gen/Zn and TA-Zn-Gen 3 NPs groups, TA/Gen/Zn or TA-Zn-Gen 3 NPs were administered at 10 mg/kg at 1 h and 12 h after CLP. “TA/Gen/Zn” is a soluble mixture of tannic acid, gentamicin, and Zn²⁺, and the dosage of each component is the same as in the TA-Zn-Gen 3 NPs.

The clinical scores, survival rate, and body weight were monitored for five consecutive days. The criteria of clinical score according to a previously established method were listed as follows: 0, no symptoms; 1, piloerection and huddling; 2, piloerection, diarrhea, and huddling; 3, lack of interest in surroundings and severe diarrhea; 4, decreased movement and listless appearance; 5, loss of self-righting reflex. Mice were humanely killed when they exhibited a score of 5.

FIG. 7A presents a schematic of CLP-induced sepsis model and treatment schedule. As shown in FIG. 7B, The TA/Gen/Zn and TA-Zn-Gen 3 NPs treatment groups exhibited significantly diminished clinical score, indicating recovery of physical state after treatment. No mice in the CLP group without treatment survived after 2 days. Repetitive intraperitoneal (i.p.) administration with TA/Gen/Zn at 1 h and 12 h post-CLP resulted in a notable survival rate (40%) (FIG. 7C). The same administration of TA-Zn-Gen 3 NPs further delayed CLP-induced lethality and produced the highest survival rate (60%). This result may be due the scavenging of multiple mediators of sepsis by the NPs and their prolonged circulation and tissue retention time, improving their protection from organ damage. Increased mouse body weight was observed after two days of treatment with TA/Gen/Zn or TA-Zn-Gen 3 NPs (FIG. 7D), further indicating an anti-sepsis therapeutic effect.

It is to be understood that the present subject matter is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the technology as defined by the appended claims.

### REFERENCES

1. Hotchkiss, R. S., Moldawer, L. L., Opal, S. M., Reinhart, K., Turnbull, I. R., and Vincent J. L. (2016). Sepsis and septic shock. Nat. Rev. Dis. Primers 2, 16045.
2. Angus, D. C., and van der Poll, T. (2013). Severe sepsis and septic shock. N. Engl. J. Med. 369, 840-851.
3. Arnold, C. News Feature: The quest to solve sepsis. (2018). Proc. Natl. Acad. Sci. U S A 115, 3988-3991.
4. Stearns-Kurosawa, D. J., Osuchowski, M. F., Valentine, C., Kurosawa, S., and Remick, D. G. (2011). The pathogenesis of sepsis. Annu. Rev. Pathol.: Mech. Dis. 6, 19-48.
5. Goldenberg, N. M., Steinberg, B. E., Slutsky, A. S., and Lee, W. L. (2011). Broken barriers: A new take on sepsis pathogenesis. Sci. Transl. Med. 3, 88ps25.
6. Beishuizen, A., Vermes, I., and Haanen, C. (1998). Endogenous mediators in sepsis and septic shock. Adv. Clin. Chem. 33, 55-131.
7. Jaber, B. L., and Pereira, B. J. G. (1996). Inflammatory mediators in sepsis: rationale for extracorporeal therapies? Am. J. Kidney Dis. 28, S35-S49.
8. Mosevoll, K. A., Skrede, S., Markussen, D. L., Fanebust, H. R., Flaatten, H. K., ABmus, J., Reikvam, H., Bruserud, and 0. (2018). Inflammatory mediator profiles differ in sepsis patients with and without bacteremia. Front. Immunol. 9, 691.
9. Harrison, C. (2010). Sepsis: calming the cytokine storm. Nat. Rev. Drug Discov. 9, 360-361.
10. Wang, H., and Ma, S. (2008). The cytokine storm and factors determining the sequence and severity of organ dysfunction in multiple organ dysfunction syndrome. Am. J Emerg. Med. 26,711-715.
11. Marsman, G., Zeerleder, S., and Luken, B. M. (2016). Extracellular histones, cell-free DNA, or nucleosomes: differences in immunostimulation. Cell Death Dis. 7, e2518-e2518.
12. Timmermans, K., Kox, M., Scheffer, G. J., and Pickkers, P. (2016) Plasma nuclear and mitochondrial dna levels, and markers of inflammation, shock, and organ damage in patients with septic shock. Shock 45, 607-612.
13. Rhodes, A., and Cecconi, M. (2012). Cell-free DNA and outcome in sepsis. Crit. Care 16, 170.
14. Lee, J., Sohn, J. W., Zhang, Y. Leong, K. W., Pisetsky, D., and Sullenger, B. A. (2011).

Nucleic acid-binding polymers as anti-inflammatory agents. Proc. Nat.1 Acad. Sci. U S A 108, 14055-14060.
15. Holl, E. K., Shumansky, K. L., Borst, L. B., Burnette, A. D., Sample, C. J., Ramsburg, E. A., and Sullenger, B. A. (2016). Scavenging nucleic acid debris to combat autoimmunity and infectious disease. Proc. Natl. Acad. Sci. U S A 113, 9728-9733.
16. Dawulieti, J., et al. (2020). Treatment of severe sepsis with nanoparticulate cell-free DNA scavengers. Sci. Adv. 6, eaay7148.
17. Liang, H., et al. (2018). Cationic nanoparticle as an inhibitor of cell-free DNA-induced inflammation. Nat. commun. 9, 4291-4291.
18. Jones, C. F., et al. (2012). Cationic PAMAM dendrimers aggressively initiate blood clot formation. ACS Nano 6, 9900-9910.
19. Ohto, U., Fukase, K., Miyake, K., and Shimizu, T. (2012). Structural basis of species-specific endotoxin sensing by innate immune receptor TLR4/MD-2. Proc. Nat.1 Acad. Sci. U S A 109, 7421.
20. Beutler, B., and Rietschel, E. T. (2003). Innate immune sensing and its roots: the story of endotoxin. Nat. Rev. Immunol. 3, 169-176.
21. Maysinger, D., Hebert, M. L., Ji, J., Jabbour, K., Dernedde, J., Silberreis, K., Haag, R., and Kriz, J. (2019). Dendritic polyglycerols are modulators of microglia-astrocyte crosstalk. Future Neurol. 14, FNL31.
22. Wittebole, X., Castanares-Zapatero, D., and Laterre, P. F. (2010). Toll-like receptor 4 modulation as a strategy to treat sepsis. Mediators Inflamm. 2010, 568396.
23. Lima, C. X., et al. (2015). Therapeutic effects of treatment with Anti-TLR2 and Anti-TLR4 monoclonal antibodies in polymicrobial sepsis. PLOS One 10, e0132336.
24. Solov'eva, T., Davydova, V., Krasikova, I., and Yermak, I. (2013). Marine compounds with therapeutic potential in gram-negative sepsis. Mar. Drugs 11, 2216-2229.
25. Cao, F., Zhang, L., You, Y., Zheng L., Ren. Ji., and Qu, X. (2020). An Enzyme-mimicking single-atom catalyst as an efficient multiple reactive oxygen and nitrogen species scavenger for sepsis management. Angew. Chem. Int. Ed. 59, 5108-5115.
26. Rittirsch, D., Flierl, M. A., and Ward, P. A. (2008). Harmful molecular mechanisms in sepsis. Nat. Rev. Immunol. 8, 776-787.
27. Hassan, F. I., Didari, T., Khan, F., Niaz, K., Mojtahedzadeh, M., and Abdollahi, M. (2020). A review on the protective effects of metformin in sepsis-induced organ failure. Cell J. 21, 363-370.
28. Dinarello, C. A. (2001). Anti-cytokine therapies in response to systemic infection. J. Invest. Dermatol. Symp. Proc. 6, 244-250.
29. Roger, T., et al. (2009). Protection from lethal Gram-negative bacterial sepsis by targeting toll-like receptor 4. Proc. Nat.1 Acad. Sci. U S A 106, 2348.
30. Fisher, C. J., et al. (1996). Treatment of Septic Shock with the Tumor Necrosis Factor Receptor:Fc Fusion Protein. N. Engl. J. Med. 334, 1697-1702.
31. Opal, S.M., et al. (1997). Confirmatory interleukin-1 receptor antagonist trial in severe sepsis: A phase III, randomized, doubleblind, placebo-controlled, multicenter trial. Crit. Care Med. 25, 1115-1124.
32. Shi, C., et al. (2020). A nanotrap improves survival in severe sepsis by attenuating hyperinflammation. Nat. Commun. 11, 3384.

What is claimed is:

1. A method of targeting multiple mediators to treat sepsis which comprises administering to a patient in need thereof an agent comprising tannic acid, Zn²⁺, and gentamicin in an amount and under conditions to provide at least two of the following:
(a) the inhibition of the nucleic acid-sensed toll-like receptor (TLR) activation is effected;
(b) the inhibition on reactive oxygen species (ROS) induced DNA damage and cell death are effected;
(c) the migration of microphage induced by activated microphage is reduced;
(d) the generation of NO induced by lipopolysaccharides (LPS) is decreased; and
(e) the immune response induced by bacteria is mitigated.

2. The method of claim 1, wherein the agent consists of tannic acid, Zn²⁺, and gentamicin.

3. The method of claim 1, wherein the agent is in the form of a nanoparticle.

4. The method of claim 1, wherein the surface charge of the agent is negatively charged.

5. The method of claim 1, wherein the nucleic acid is pathogen-derived or is released from dead or damaged cells of the patient.

6. The method of claim 1, wherein the TLR comprises TLR3, TLR9, or both.

7. The method of claim 1, wherein the agent binds the nucleic acid independent of the sequences, structure or chemistry of the nucleic acid.

8. The method of claim 1, wherein the ROS is ROS generated in an inflammatory site of the patient.

9. The method of claim 1, wherein the macrophage is from the patient.

10. The method of claim 1, wherein the LPS derived from the gram-negative bacteria.

11. The method of claim 10, wherein the gram-negative bacteria is Escherichia coli.

12. The method of claim 1, wherein the immune response induced by bacteria is by gram-negative bacteria.

13. The method of claim 1, wherein the patient was exposed to a nucleic acid/ROS/LPS/bacteria prior to administering of the agent or further comprising exposing the patient to a nucleic acid/ROS/LPS/bacteria prior to administering the agent.

14. The method of claim 6, further comprising detecting the inhibition of activation of TLR3 or TLR9 by measuring TNF-α or IL-6 production in the patient.

15. The method of claim 6, further comprising detecting the inhibition of activation of TLR3 or TLR9 using reporter cells involving poly (I:C) or CpG.

16. The method of claim 1, wherein administration of the agent results in a mitigation in the acute inflammatory response in the patient.

17. The method of claim 1, wherein the patient suffers from a disease selected from the group consisting of rheumatoid arthritis, spinal cord injury, psoriasis, systemic lupus erythematosus, inflammatory bowel disease, traumatic brain injury, an infectious disease, a cardiovascular disease, cancer bacterial sepsis, multiple sclerosis, chronic obstructive pulmonary disease, and obesity.

18. The method of claim 1, wherein the ROS comprises one or both of hydroxyl radicals (·OH) and superoxide radicals (O₂^{·-}).

19. The method of claim 1, wherein the macrophage is RAW 264.7.

20. The method of claim 19, wherein the agent inhibits TNF-α release from RAW 264.7 cells.
